(19) United States
(12) Patent Application Publication
(10) Pub. No.: US 2025/0299803 A1
(43) Pub. Date: Sep. 25, 2025

(54) SYSTEMS AND METHODS TO PROCESS ELECTRONIC IMAGES FOR DETERMINING TREATMENT

(71) Applicant: PAIGE.AI, Inc., New York, NY (US)

(72) Inventors: Jeremy Daniel KUNZ, New York, NY (US); Dilip THIAGARAJAN, New York, NY (US)

(21) Appl. No.: 19/232,144

(22) Filed: Jun. 9, 2025

Related U.S. Application Data
(63) Continuation of application No. 18/049,220, filed on Oct. 24, 2022.
(60) Provisional application No. 63/262,979, filed on Oct. 25, 2021.

(51) Int. Cl. G16H 30/40; G06N 5/04; G06T 7/00; G16H 50/20

(52) U.S. Cl. CPC G16H 30/40; G06N 5/04; G06T 7/0016; G16H 50/20; G06T 2207/20081; G06T 2207/30024; G06T 2207/30096

(57) ABSTRACT

A computer-implemented method for processing digital pathology images, the method including receiving a plurality of digital pathology images of at least one pathology specimen, the pathology specimen being associated with a patient. The method may further include determining receiving metadata corresponding to the plurality of digital pathology images, the metadata comprising data regarding previous medical treatment of the patient. Next, the method may include providing the medical images and metadata as input to a machine learning system, the machine learning system having been trained by receiving as input historical treatment information and digital images labeled with a predicted treatment regimen. Lastly, the method may include outputting, by the machine learning system, a treatment effectiveness assessment.

### RELATED APPLICATION(S)

This application is a continuation of and claims the benefit of priority to U.S. application Ser. No. 18/049,220, filed on Oct. 24, 2022, which claims priority to U.S. Provisional Application No. 63/262,979 filed Oct. 25, 2021, each of which are incorporated herein by reference in their entirety.

### FIELD OF THE DISCLOSURE

Various embodiments of the present disclosure pertain, generally, to processing electronic images to assess treatment for an individual. More specifically, particular embodiments of the present disclosure relate to systems and methods for using artificial intelligence to treatment assessments over time for one or more users.

### BACKGROUND

The level of treatment for one or more diseases may vary depending on one or more factors such as the severity of a given disease. Accordingly, a correct dosage of treatment (e.g., medicine, medical treatment, etc.) may be important to ensure that, for example, a disease responds to the treatment. However, many treatments can have deleterious effects on a patient. For example, in radiotherapy for head and neck cancer, too little treatment may fail to cure a disease. Additionally, though overtreatment may cure a given disease, it may result in unexpected effects such as the loss of teeth and other facial features. For treatment of many cancers, there are often multiple drugs given simultaneously. For example, in estrogen-receptor-positive (ER+) breast cancer, both chemotherapy and endocrine therapy may be given to a patient. Determining the right level of both chemotherapy and endocrine may be important to obtaining the best outcome.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the present disclosure, systems and methods are disclosed for processing electronic images. In one aspect, a computer-implemented method for processing electronic medical images to assess treatment for an individual is disclosed. The method may comprise receiving a plurality of medical images of at least one pathology specimen, the pathology specimen being associated with a patient; receiving metadata corresponding to the plurality of digital pathology images, the metadata comprising data regarding previous medical treatment of the patient; providing the medical images and metadata as input to a machine learning system, the machine learning system having been trained by receiving as input historical treatment information and digital images labeled with a predicted treatment regimen; and outputting, by the machine learning system, a treatment effectiveness assessment.

In another aspect, a system for processing electronic digital medical images may comprise at least one memory storing instructions and at least one processor configured to execute the instructions to perform operations. The at least one processor may comprise receiving a plurality of medical images of at least one pathology specimen, the pathology specimen being associated with a patient; receiving metadata corresponding to the plurality of digital pathology images, the metadata comprising data regarding previous medical treatment of the patient; providing the medical images and metadata as input to a machine learning system, the machine learning system having been trained by receiving as input historical treatment information and digital images labeled with a predicted treatment regimen; and outputting, by the machine learning system, a treatment effectiveness assessment.

In another aspect, a non-transitory computer-readable medium storing instructions that, when executed by a processor, perform operations processing electronic digital medical images, is disclosed. The operations may include receiving a plurality of medical images of at least one pathology specimen, the pathology specimen being associated with a patient; receiving metadata corresponding to the plurality of digital pathology images, the metadata comprising data regarding previous medical treatment of the patient; providing the medical images and metadata as input to a machine learning system, the machine learning system having been trained by receiving as input historical treatment information and digital images labeled with a predicted treatment regimen; and outputting, by the machine learning system, a treatment effectiveness assessment.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A illustrates an exemplary block diagram of a system and network for processing images, according to techniques presented herein.

FIG. 1B illustrates an exemplary block diagram of a tissue viewing platform according to techniques presented herein.

FIG. 1C illustrates an exemplary block diagram of a slide analysis tool, according to techniques presented herein.

FIG. 2 illustrates a process for determining a treatment of an individual based on one or more digital images, according to techniques presented herein.

FIG. 3A is a flowchart illustrating how to train an algorithm for image region detection, according to techniques presented herein.

FIG. 3B is a flowchart illustrating methods for image region detection, according to one or more exemplary embodiments herein.

FIG. 4 is a flowchart illustrating an exemplary process for using a trained system for outputting an embedding, according to techniques presented herein.

FIG. 5A is flowchart illustrating an example method for training a system to receive one or more modalities and output an embedded vector representation, according to techniques presented herein.

FIG. 5B is a flowchart illustrating an example method for using a system to receive one or more modalities and output an embedded vector representation, according to techniques presented herein.

FIG. 6 is a flowchart illustrating an exemplary process for using a trained system to determine a treatment effectiveness based on one or more digital pathology images, according to techniques presented herein.

FIG. 7A is a flowchart illustrating an example method for training a system to determine a treatment effectiveness based on one or more a digital pathology image, according to techniques presented herein.

FIG. 7B is a flowchart illustrating an example method for using a system to determine a treatment effectiveness based on one or more a digital pathology image, according to techniques presented herein.

FIG. 8 is a flowchart illustrating an exemplary process for using a trained system to determine a treatment recommendation based on one or more digital pathology images, according to techniques presented herein.

FIG. 9A is a flowchart illustrating an example method for training a system to determine a treatment recommendation based on one or more a digital pathology image, according to techniques presented herein.

FIG. 9B is a flowchart illustrating an example method for using a system to determine a treatment recommendation based on one or more a digital pathology image, according to techniques presented herein.

FIG. 10 is a flowchart illustrating an exemplary process for using a trained system to determine a treatment recommendation based on one or more digital pathology images, according to techniques presented herein.

FIGS. 11A-11C provides exemplary user interfaces for the system, allowing for one or more users to set a dosage treatment, according to techniques presented herein.

FIG. 12 is a flowchart illustrating an example method for determining a treatment recommendation for one or more users.

FIG. 13 depicts an example of a computing device that may execute techniques presented herein, according to one or more embodiments.

### DESCRIPTION OF THE EMBODIMENTS

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

As used herein, a “machine learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Deep learning techniques may also be employed. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

Embodiments of the disclosed subject matter are directed to applying artificial intelligence (AI)/machine learning (ML) models to determining and/or adjusting treatment, treatment effectiveness, and/or treatment dosages. Disclosed herein are AI systems for inferring the effectiveness of treatment in terms of disease eradication and damage to healthy tissue. Also disclosed are AI systems for recommending treatment dosages. Also disclosed are AI systems for recommending changes in treatment regimen.

In clinical practice, determining a correct treatment type and treatment amount for a patient may be challenging. In particular, determining an effective treatment for a previously untreated patient might be difficult, especially when the treatment determination is determined based on the analysis of digital medical images (e.g., histopathological slides sampled from the patient). Techniques disclosed herein may support such determining by, for example, recommending amounts/dosages of a single or potential combination of treatments (e.g., drugs, medical interventions, etc.) for treating an untreated patient based on one or more digital medical images. Additionally, techniques disclosed herein may support such forecasting by, for example, assessing the successfulness/response of a treatment method and recommending an updated form of treatment for a treated patient based on a digital medical image.

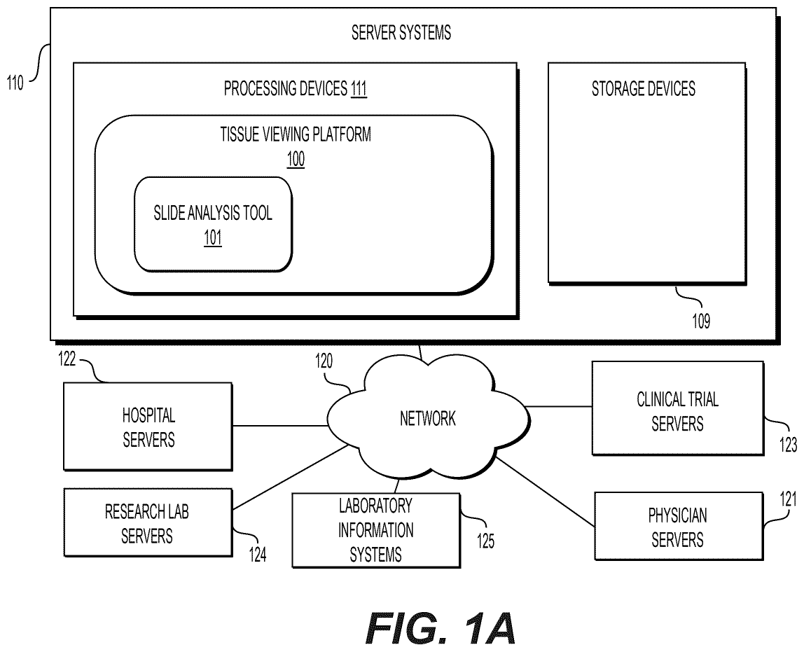

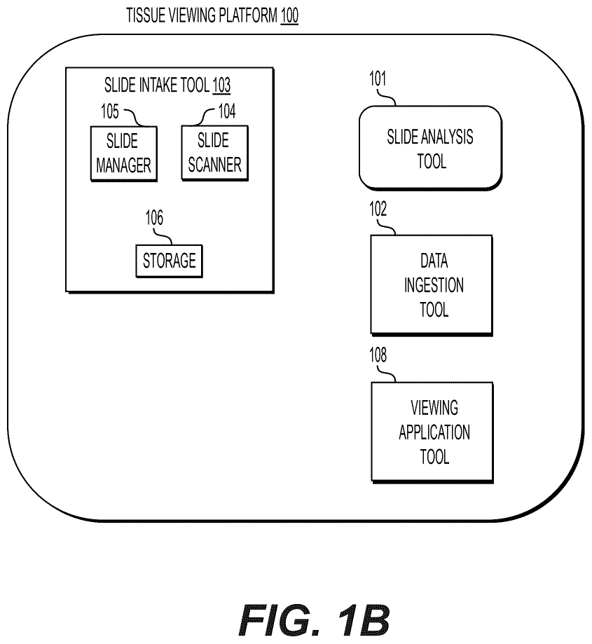

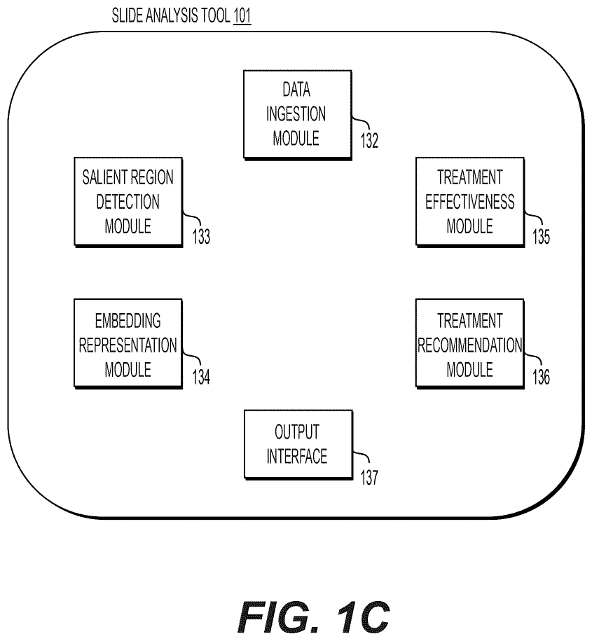

FIG. 1A illustrates a block diagram of a system and network for processing images, using machine learning, according to an exemplary embodiment of the present disclosure.

Specifically, FIG. 1A illustrates an electronic network 120 that may be connected to servers at hospitals, laboratories, and/or doctors' offices, etc. For example, physician servers 121, hospital servers 122, clinical trial servers 123, research lab servers 124, and/or laboratory information systems 125, etc., may each be connected to an electronic network 120, such as the Internet, through one or more computers, servers, and/or handheld mobile devices. According to an exemplary embodiment of the present disclosure, the electronic network 120 may also be connected to server systems 110, which may include processing devices that are configured to implement a tissue viewing platform 100, which includes a slide analysis tool 101 for determining specimen property or image property information pertaining to digital pathology image(s), and using machine learning to determine a treatment or a treatment's effectiveness for one or more individuals, according to an exemplary embodiment of the present disclosure.

The physician servers 121, hospital servers 122, clinical trial servers 123, research lab servers 124, and/or laboratory information systems 125 may create or otherwise obtain images of one or more patients' cytology specimen(s), histopathology specimen(s), slide(s) of the cytology specimen(s), digitized images of the slide(s) of the histopathology specimen(s), or any combination thereof. The physician servers 121, hospital servers 122, clinical trial servers 123, research lab servers 124, and/or laboratory information systems 125 may also obtain any combination of patient-specific information, such as age, medical history, cancer treatment history, family history, past biopsy or cytology information, etc. The physician servers 121, hospital servers 122, clinical trial servers 123, research lab servers 124, and/or laboratory information systems 125 may transmit digitized slide images and/or patient-specific information to server systems 110 over the electronic network 120. Server systems 110 may include one or more storage devices 109 for storing images and data received from at least one of the physician servers 121, hospital servers 122, clinical trial servers 123, research lab servers 124, and/or laboratory information systems 125. Server systems 110 may also include processing devices for processing images and data stored in the one or more storage devices 109. Server systems 110 may further include one or more machine learning tool(s) or capabilities. For example, the processing devices may include a machine learning tool for a tissue viewing platform 100, according to one embodiment. Alternatively or in addition, the present disclosure (or portions of the system and methods of the present disclosure) may be performed on a local processing device (e.g., a laptop).

The physician servers 121, hospital servers 122, clinical trial servers 123, research lab servers 124, and/or laboratory information systems 125 refer to systems used by pathologists for reviewing the images of the slides. In hospital settings, tissue type information may be stored in one of the laboratory information systems 125. However, the correct tissue classification information is not always paired with the image content. Additionally, even if a laboratory information system is used to access the specimen type for a digital pathology image, this label may be incorrect due to the face that many components of a laboratory information system may be manually input, leaving a large margin for error. According to an exemplary embodiment of the present disclosure, a specimen type may be identified without needing to access the laboratory information systems 125, or may be identified to possibly correct laboratory information systems 125. For example, a third party may be given anonymized access to the image content without the corresponding specimen type label stored in the laboratory information system. Additionally, access to laboratory information system content may be limited due to its sensitive content.

FIG. 1B illustrates an exemplary block diagram of a tissue viewing platform 100 for determining specimen property of image property information pertaining to digital pathology image(s), using machine learning. For example, the tissue viewing platform 100 may include a slide analysis tool 101, a data ingestion tool 102, a slide intake tool 103, a slide scanner 104, a slide manager 105, a storage 106, and a viewing application tool 108.

The slide analysis tool 101, as described below, refers to a process and system for processing digital images associated with a tissue specimen, and using machine learning to analyze a slide, according to an exemplary embodiment.

The data ingestion tool 102 refers to a process and system for facilitating a transfer of the digital pathology images to the various tools, modules, components, and devices that are used for classifying and processing the digital pathology images, according to an exemplary embodiment.

The slide intake tool 103 refers to a process and system for scanning pathology images and converting them into a digital form, according to an exemplary embodiment. The slides may be scanned with slide scanner 104, and the slide manager 105 may process the images on the slides into digitized pathology images and store the digitized images in storage 106.

The viewing application tool 108 refers to a process and system for providing a user (e.g., a pathologist) with specimen property or image property information pertaining to digital pathology image(s), according to an exemplary embodiment. The information may be provided through various output interfaces (e.g., a screen, a monitor, a storage device, and/or a web browser, etc.).

The slide analysis tool 101, and each of its components, may transmit and/or receive digitized slide images and/or patient information to server systems 110, physician servers 121, hospital servers 122, clinical trial servers 123, research lab servers 124, and/or laboratory information systems 125 over an electronic network 120. Further, server systems 110 may include one or more storage devices 109 for storing images and data received from at least one of the slide analysis tool 101, the data ingestion tool 102, the slide intake tool 103, the slide scanner 104, the slide manager 105, and viewing application tool 108. Server systems 110 may also include processing devices for processing images and data stored in the storage devices. Server systems 110 may further include one or more machine learning tool(s) or capabilities, e.g., due to the processing devices. Alternatively or in addition, the present disclosure (or portions of the system and methods of the present disclosure) may be performed on a local processing device (e.g., a laptop).

Any of the above devices, tools and modules may be located on a device that may be connected to an electronic network 120, such as the Internet or a cloud service provider, through one or more computers, servers, and/or handheld mobile devices.

FIG. 1C illustrates an exemplary block diagram of a slide analysis tool 101, according to an exemplary embodiment of the present disclosure. The slide analysis tool 101 may include a data ingestion module 132, a salient region detection module 133, an embedding representation module 134, a treatment effectiveness module 135, a treatment recommendation module 136, and an output interface 137. All modules within the slide analysis tool 101 may be capable of receiving information from any one or more of the server systems 110, physician servers 121, hospital servers 122, clinical trial servers 123, research lab servers 124, and/or laboratory information systems 125. Images used for training may come from real sources (e.g., humans, animals, etc.) or may come from synthetic sources (e.g., graphics rendering engines, 3D models, etc.).

The data ingestion module 132, as described in greater detail below, may refer to a process and system for receiving digital medical images/pathology slides (e.g., digitalized images of a slide-mounted history or cytology specimens), and additional information relating to one or more patients. Examples of digital pathology images may include (a) digitized slides stained with a variety of stains, such as (but not limited to) H&E, Hematoxylin alone, IHC, molecular pathology, etc.; and/or (b) digitized image samples from a 3D imaging device, such as micro-CT. Further, the data ingestion module 132 may be capable of receiving metadata in the forms of text. The data ingestion module 132 may, for instance, receive data from the data ingestion tool 102.

The salient region detection module 133, as described in detail below, may refer to system and processes for identifying images or specific regions of images relevant to the system. The overall system may then only perform analysis on the salient regions.

The embedding representation module 134, as described in detail below, may refer to a system capable of receiving sequences of clinical data for one or more patients and output one or more embedding representing the conditions of the one or more patients. The embedding representation module 134 may receive information from the data ingestion module 132 and/or the salient region detection module 133 in additional to information received through network 120 or storage devices 109. The embedding representation module 134 may output the received data as one or more embeddings. Further, the embedding representation module 134 may be capable of determining/inferring missing data points for later usage in the system as described in detail below.

The treatment effectiveness module 135, as described in detail below, may refer to a trained system capable of measuring the effectiveness of one or more treatments on a patient over time. The trained system may receive digital medical images at one or more periods of time and then determine the effectiveness of the one or more treatments. In some examples of the system, the treatment effectiveness module 135 may receive digital medical images. In another example of the system, the treatment effectiveness module 135 may receive as input embeddings outputted by the embedding representation module 134.

The treatment recommendation module 136, as described in greater detail below, may be capable of training and using a machine learning system that assess one or more digital medical image to recommend a treatment regimen for one or more patients (e.g., the frequency and amount/dosage of a single or potential combination of drugs/treatments). In some examples of the system, the treatment recommendation module 136 may receive digital medical images. In another example of the system, the treatment recommendation module 136 may receive as input embeddings outputted by the embedding representation module 134.

The output interface 137 may be used to output information about the inputted images and additional information (e.g., to a screen, monitor, storage device, web browser, etc.). The output information may include information related to the effectiveness of prior treatments and/or treatment recommendations for one or more patients. Further, output interface 137 may output WSI's that indicate locations/salient regions that include evidence related to outputs from the treatment effectiveness module 135 and treatment recommendation module 136. The output interface 137 may be capable of outputting treatment recommendations and treatment effectiveness to the viewing application tool 108.

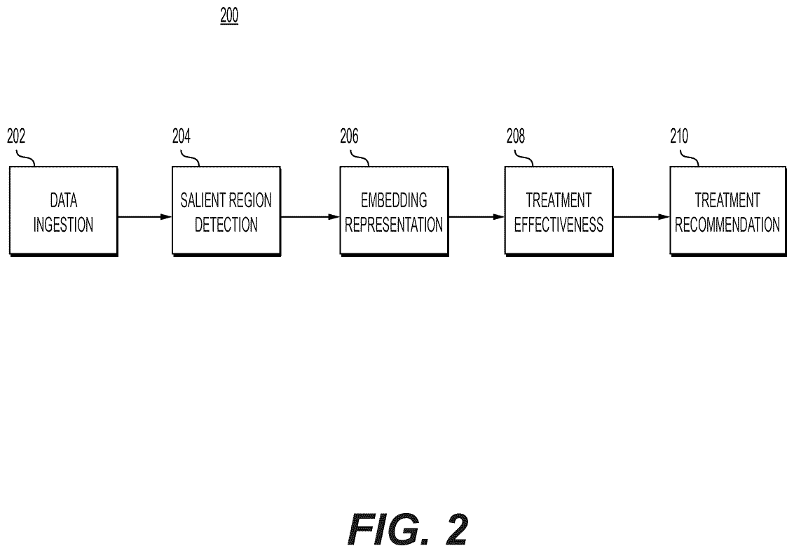

FIG. 2 illustrates a process for measuring the effectiveness of a treatment over time and/or determining a treatment for one or more patients by analyzing one or more digital medical images, according to techniques presented herein. Flowchart 200 may include techniques that may be implemented by using a data ingestion module 132, a salient region detection module 133, a universal or multimodal embedding representation module 134 for a patient, a treatment effectiveness module 135, and/or a treatment recommendation module 136 as will be discussed in greater detail below.

At step 202, the system (e.g., the data ingestion module 132) may receive data such as one or more digital medical images. The digital medical images may include untreated or treated digital whole slide image (WSI) by chemotherapy, radiation therapy etc., magnetic resonance imaging (MRI), computed tomography (CT), positron emission tomography (PET), mammogram, etc.). The digital medical images may be stored on a digital storage device 109 (e.g., hard drive, network drive, cloud storage, RAM, etc.). Additionally, at step 202, metadata related to the digital medical images may be received such as the date and time of when the medical specimen of the digital medical images were sampled. The metadata may further include information as to whether the particular digital medical images were treated or untreated slides. Additional information may also be ingested such as age, ethnicity, ancillary test results, etc. The metadata may further include information related to treatments that may have been administered to a patient prior to the medical specimen being sampled. The information may be provided in multiple forms including total dosages given prior to tissue removal, or individual treatments given over time prior to tissue removal. Time before surgery may also be received as input. Exemplary metadata received with digital medical images may include a number of days between treatment and tissue removal, and/or time intervals of treatments.

Further, the system may ingest information that corresponds particular metadata to inputted digital medical images. This may allow for training/using of an applicable machine learning system or component as discussed in greater detail below, as each image may be paired with available drug dosage information. Metadata may further include input information from a hospital information system such as radiation, chemotherapy, or other treatment information may be received. Such input information may be provided in multiple forms: e.g., total dosages given prior to tissue removal, or individual treatments given over time prior to tissue removal.

At step 204, the system may perform salient region detection (e.g., by the salient region detection module 133) on the one or more digital medical images received at step 202. This process may be implemented manually or automatically using artificial intelligence. A salient region detection module 133, as further described below, may be used to identify the salient regions to be analyzed for each digital image. A salient region may be defined as an image or area of an image that is considered relevant to a pathologist performing diagnosis of an image. A digital image may be divided into patches/tile and a score may be associated with each tile, wherein the score indicates how relevant a particular tile/patch is to a particular task. Patches/tiles with scores above a threshold value may then be considered salient regions. In one example, a salient region of a slide may refer to the tissue areas, in contrast to the rest of the slide, which may be the background area of the WSI. One or more salient regions may be identified and analyzed for each digital image. An entire image, or alternatively specific regions of an image, may be considered salient. The salient regions may be identified by one or more software modules. Salient region determination techniques are discussed in U.S. application Ser. No. 17/313,617, which is incorporated by reference herein in its entirety.

At step 206, a universal or multimodal embedding representation module may be implemented (e.g., by the embedding representation module 134). As will be discussed in greater detail below, the system may receive a sequence of clinical data for a patient for a fixed number of modalities (e.g., a H&E WSI, a IHC WSI, a CT scan, patient synoptic report, and/or information related to treatment). This may include the digital medical images and corresponding metadata from step 202. Further, the salient region detection module 133 may be applied to the inputted images prior to the universal or multimodal embedding representation module receives the digital medical images. At a particular time, not all modalities of data might be available from the fixed set of modalities consider. For example, the system may receive an H&E WSI and the treatment information, however, the system may not have access to the patient synoptic report or a CT scan. The embedding representation module 134 may convert all received data into a representative embedding that may be used for downstream tasks. This may allow for the treatment effectiveness module 135 and the treatment recommendation module 136 to receive standardized data from the embedding representation module 134. Further, the embedding representation module 134 may be capable of determining missing data, for example, by using a generative approach to interpolate between two time points. Data may be handled in sequence, such as by using a recurrent neural network (RNN) or transformer model.

At step 208, the system may determine a previous treatment's effectiveness for one or more patients (e.g., using the treatment effectiveness module 135). As will be discussed in greater detail below, this module may measures the effectiveness of a treatment over time in two capacities: 1) how much the diseased tissue is eradicated, shrunk, or shows signs of being cured, and 2) how much healthy tissue has been damaged by the treatment. The system may create a score for each of the two capacities and an overall score to measure the effectiveness of the previous treatment.

At step 210, the system may implement a treatment recommendation module (e.g., treatment recommendation module 136). Given an image of a slide or a set of salient region images from a slide, recommend frequency and amounts/dosages of a single or potential combination of drugs (e.g., from a known set of drugs used to treat the particular tissue type being analyzed) may be provided for a patient from whom the slide was obtained. If the slide is specified as treated, the treatment regimen may be received (frequency and amounts/dosages of a single or potential combination of drugs) and new frequency and/or amounts/dosages of a single or potential combination of drugs may be recommended. This module may incorporate spatial information from disparate regions in an image. The prediction may be output to an electronic storage device 109 or displayed through the output interface 137 (e.g., a screen, a monitor, and/or a web browser, etc.).

As previously mentioned, at step 204, the system may utilize a salient region detection module 133 to determine salient regions of the inputted digital medical images. The salient region detection module 133 may assign a continuous score of interest to a digital medical image or to an area of a digital medical image to quantify whether a region is salient. A continuous score of interest may be specific to certain structures within a digital image, and it may be beneficial to identify relevant regions so that they may be included while excluding irrelevant ones. For example, with MRI, PET, or CT, data localizing a specific organ of interest may be needed and thus the specific organs may receive a higher continuous score of interest. Salient region identification may cause a downstream machine learning system to learn how to detect morphologies from less annotated data and to make more accurate predictions.

The salient region detection module 133 may output a salient region specified by an annotator using an image segmentation mask, a bounding box, line segment, point annotation, freeform shape, or a polygon, or any combination of the same. Alternatively, this module may be generated using machine learning to identify the appropriate locations.

There may be two exemplary approaches to using machine learning to create a salient region detector. These approaches may include strongly supervised methods that identify precisely where the morphology of interest could be found and weakly supervised methods that do not provide a precise location.

Strongly supervised training may be implemented by using an image and location of salient regions that could potentially express a biomarker, as input. For 2D images, e.g., WSI in pathology, these locations could be specified with pixel-level labeling, bounding box-based labeling, polygon-based labeling, or using a corresponding image where the saliency has been identified (e.g., using IHC). For 3D images, e.g., CT and MRI scans, the locations could be specified with voxel-level labeling, using a cuboid, etc. or use a parameterized representation which may allow for subvoxel-level labeling, such as parameterized curves or surfaces, or deformed template(s). Weakly supervised training may be implemented using the image or images and the presence/absence of the salient regions, but the exact location of the salient location may not be specified.

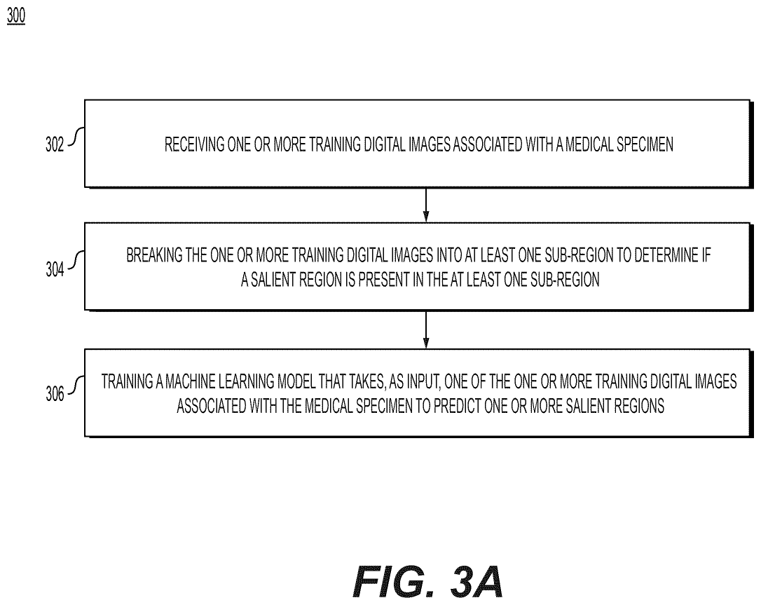

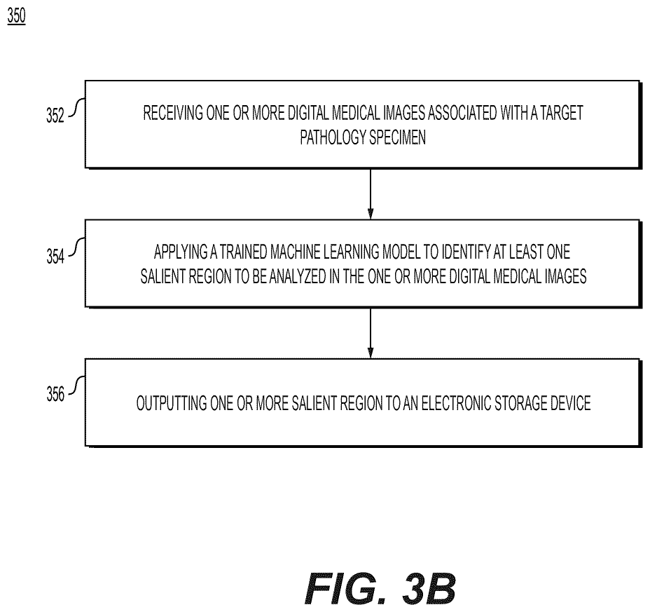

FIG. 3A is a flowchart illustrating an example of how to train an algorithm for salient region detection module 133, according to techniques presented herein. The processes and techniques described in FIG. 3A may be used to train a machine learning model to identifier salient regions of medical digital images. The method 300 of FIG. 3A depicts steps that may be performed by, for example, the salient region detection module 133 of slide analysis tool 101 as described above in FIG. 1C. Alternatively, the method may be performed by an external system.

Flowchart/method 300 depicts training steps to train a machine learning model as described in further detail in steps 302-306. The machine learning model may be used to identify salient regions of digital medical images as discussed further below.

At step 302, the system (e.g., the salient region detection module 133) may receive one or more digital images of a medical specimen (e.g., from histology, CT, MRI, etc.) into a digital storage device (e.g., hard drive, network drive, cloud storage, RAM, etc.) and receive an indication of a presence or absence of a salient region (e.g., invasive cancer present, LVSI, in situ cancer, etc.) within the one or more images.

At step 304, each digital image may be broken into sub-regions that may then have their saliency determined. Sub-regions may be specified in a variety of methods and/or based on a variety of criteria, including creating tiles of the image, segmentations based on edge/contrast, segmentations via color differences, segmentations based on energy minimization, supervised determination by the machine learning model, EdgeBoxes, etc.

At step 306 a machine learning system may be trained that takes as input a digital image and predicts whether the salient region is present or not. Training the salient region detection module may also include training a machine learning system to receive, as an input, a digital image and to predict whether the salient region is present or not. Many methods may be used to learn which regions are salient, including but not limited to weak supervision, bounding box or polygon-based supervision, or pixel-level or voxel-level labeling.

Weak supervision may involve training a machine learning model (e.g., multi-layer perceptron (MLP), convolutional neural network (CNN), transformers, graph neural network, support vector machine (SVM), random forest, etc.) using multiple instance learning (MIL). The MIL may use weak labeling of the digital image or a collection of images. The label may correspond to the presence or absence of a salient region.

Bounding box or polygon-based supervision may involve training a machine learning model (e.g., R-CNN, Faster R-CNN, Selective Search, etc.) using bounding boxes or polygons. The bounding boxes or polygons may specify sub-regions of the digital image that are salient for detection of the presence or absence of a biomarker.

Pixel-level or voxel-level labeling (e.g., semantic or instance segmentation) may involve training a machine learning model (e.g., Mask R-CNN, U-Net, fully convolutional neural network, transformers, etc.) where individual pixels and/or voxels are identified as being salient for the detection of continuous score(s) of interest. Labels could include in situ tumor, invasive tumor, tumor stroma, fat, etc. Pixel-level/voxel-level labeling may be from a human annotator or may be from registered images that indicate saliency.

FIG. 3B is a flowchart illustrating methods for how to provide image region detection, according to one or more exemplary embodiments herein. FIG. 3B may illustrate a method that utilizes the neural network that was trained in FIG. 3A. The exemplary method 350 (e.g., steps 352-356) of FIG. 3B depicts steps that may be performed by, for example, by the salient region detection module 133 of slide analysis tool 101. These steps may be performed automatically or in response to a request from a user (e.g., physician, pathologist, etc.). Alternatively, the method described in flowchart 350 may be performed by any computer process system capable of receiving image inputs such as device 1300 and capable of including or importing the neural network described in FIG. 3A.

At step 352, a system (e.g., the salient region detection module 133) may receive one or more digital medical images of a medical specimen into a digital storage device (e.g., hard drive, network drive, cloud storage, RAM, etc.). Using the salient region detection module may optionally include breaking or dividing each digital image into sub-regions and determining a saliency (e.g., cancerous tissue for which the biomarker(s) should be identified) of each sub-region using the same approach from training step 304.

At step 354, the trained machine learning system from FIG. 3A may be applied to the inputted images to predict which regions of the one or more images are salient and could potentially exhibit the continuous score(s) of interest (e.g., cancerous tissue). Applying the trained learning system to the image may include expanding the region or regions to additional tissue, such as by detecting an invasive tumor region, determining its spatial extent, and extracting a stroma around the invasive tumor.

At step 356, if salient regions are found at step 354, the system may identify the salient region locations and flag them. If salient regions are present, detection of the region can be done using a variety of methods, including but not restricted to: running the machine learning model on image sub-regions to generate the prediction for each sub-region; or using machine learning visualization tools to create a detailed heatmap, etc. Example techniques are described in U.S. application Ser. No. 17/016,048, filed Sep. 9, 2020, and Ser. No. 17/313,617, filed May 6, 2021, which are incorporated herein by reference in their entireties. The detailed heatmap may be created by using class activation maps, GradCAM, etc. Machine learning visualization tools may then be used to extract relevant regions and/or location information.

The outputted salient regions from step 356, may then be fed downstream into either the embedding representation module 134, the treatment effectiveness module 135, and/or the treatment recommendation module 136.

In multi-modal settings (e.g., when the embedding representation module 134 receives more than one type of medical images), the salient region detection module 133 may determine a salient region independently for each modality (e.g., one for WSI, one for MRIs, one for CTs, etc.).

At step 206, the system may utilize an embedding representation module 134 to normalize the data received by the data ingestion module 132 and/or the salient region detection module 133. The embedding representation module 134 may be capable of inferring missing data and outputting uniform data (e.g., one or more vectors) to the treatment effectiveness module 135 and/or the treatment recommendation module 136.

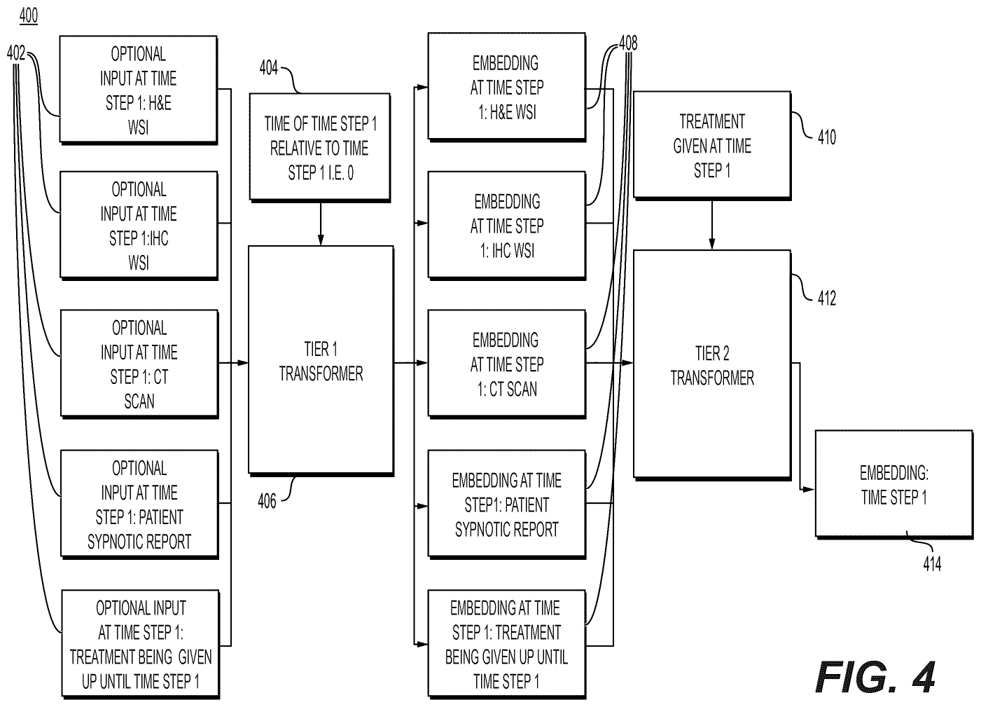

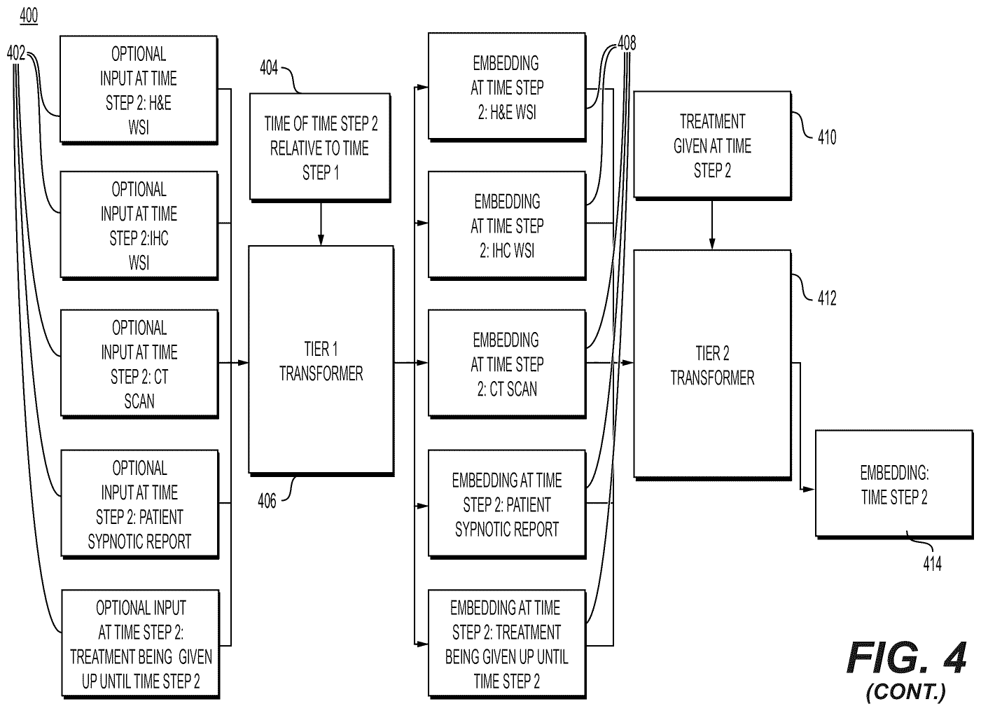

According to implementations of the embedding representation module, one or more modalities may be received as input(s). A modality may refer to any type of input data received by the system such as a digital medical image, a synoptic report, and/or information relating to treatment. FIG. 4 is a flowchart illustrating an exemplary process for using a trained system for outputting an embedding, according to techniques presented herein. Each modality may have a salient region detection module to identify relevant regions within the modality. This may be beneficial because when operating over time to determine treatment effectiveness for a patient, it may not be feasible to acquire samples from the patient for some modalities on a frequent basis. For example, CT scans may expose a patient to radiation, which needs to be minimized, and biopsies may be invasive operations that require time to heal. The embedding representation module may “fill in” missing modalities when all of them are not present at all time points.

Multi-modal information may be transformed into a single vector embedding using one or more techniques. Such techniques are disclosed herein based on transformers. Techniques disclosed herein may also be used for a single modality if the system is not trained to handle more than one modality. FIG. 4 includes an exemplary embedding representation module 134 that includes five input modalities 402 across two time steps. FIG. 4 further shows output embeddings 408 for each time step. FIG. 4 provides an example of a universal embedding representation module used for consuming slides, synoptic, and treatment data. These techniques may be extended to consume a variable number of time steps of data, and the model may be configured to be trained on any number of inputs/modalities 402.

A first tier (e.g., tier 1 transformer 406) may receive information from each modality 402 at a given time-step 404, and may turn each modality into an embedding 408 within the network. Additionally, the tier 1 transformer 406 may receive the time of the time-step 404 relative to the desired initial time-step. The desired initial time-step may be a period of time when a new treatment began, when a new dosage for a prior treatment began, or a specific time when modalities information was received. Each of the inputs/modalities 402 other than time-step 404 may be optional and/or not inputted into the tier 1 transformer 406. The network (e.g., transformers 406 and 412) may be capable of inferring non-inputted modalities 402. The second tier (e.g., tier 2 transformer 412) may then receive all the embeddings 408 from a given time-step for all modalities 402, as well as the treatment given at that time-step 410, and output an embedding 414 representing the condition as depicted by all the input modalities at each time step.

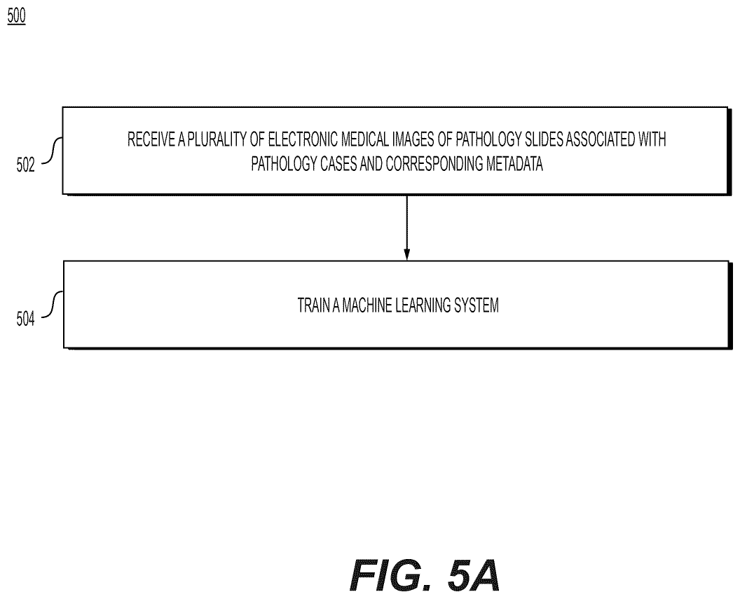

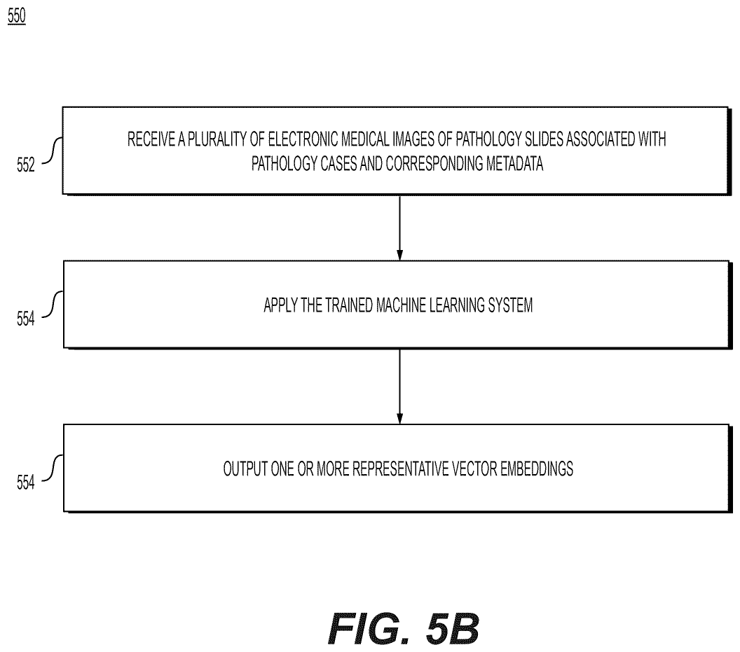

FIG. 5A is flowchart illustrating an example method for training a system to receive one or more modalities and output an embedded vector representation, according to techniques presented herein. The method 500 of FIG. 5A depicts steps that may be performed by, for example, the embedding representation module 134. Alternatively, the method 500 may be performed by an external system, where the trained system may be provided to the embedding representation module 134 for implementation.

At step 502, a plurality of training datasets may be received. The datasets may include one or more digital medical images of a medical specimen (e.g. histology, CT, MRI, etc.) for one or more individuals at different intervals of time. This may be saved into a digital storage device (e.g., hard drive, network drive, cloud storage, RAM, etc.). Further, the system may receive metadata corresponding to each individual's set of digital medical images that characterize the patient's health at different points in time. The metadata may further include all information related to treatment for the individual, such as type of treatment, time that treatment occurred (e.g., dates used, times treatment occurred, date treatment began, and/or date treatment ended), and treatment dosage amount. Additionally the metadata may contain information about the individual such as age and sex. The training datasets may also include patient synoptic reports that correspond to the individual's digital medical images.

At step 504, the system may be trained using one or more of the datasets. The trained system may be a trained machine learning (ML) system. The ML system may receive as input, the images provided as well as the metadata at each specified time point from step 502. Further, the trained system may output a quantitative representation of the patient's health at a most recent time point. This output may be in the form a single vector for each patient. The embedding representation module 134 may be trained to determine approximate times of acquisition. Given that the time points may not be equally spaced apart, the system may be provided relative time offset augmentations to determine at least an approximate time of acquisition. Many unsupervised methods may be used to learn this representation, including but not limited to a mask language model and a net time point prediction. The masked language model may mask modalities of data at random over any of the specified time points, and train the model to interpolate all data that was masked modalities of data. The next time point prediction may use the data up to (but not including) a time point, the model may be then be trained to predict all modalities of data for that selected time point.

All expected modalities of data might not be present at each time point. If a modality of data is missing at any given time point, it may be replaced with a generic missing token that can be processed by the machine learning system.

With respect to training and/or using the embedding representation module 134, image data and textual data may be handled simultaneously. For example, when training with the masked language model, images may be handled separately from text in the earlier parts of the system. Additionally, for images, the model may be trained to predict randomly masked portions of an image, whereas for texts, the model may be trained to predict randomly masked tokens of the text.

FIG. 5B is a flowchart illustrating an example method for using a system to receive one or more modalities and output an embedded vector representation, according to techniques presented herein. The exemplary method 550 (e.g., steps 552-556) of FIG. 5B depicts steps that may be performed by, for example, the embedding representation module 134. These steps may be performed automatically or in response to a request from a user (e.g., a pathologist, a department or laboratory manager, an administrator, etc.). Alternatively, the method 550 may be performed by any computer process system capable of receiving image inputs such as device 1300 and capable of storing and executing the trained system described in FIG. 5A.

At step 552, the system may receive one or more digital medical images of a medical specimen (e.g., histology, CT, MRI, etc.) for a patient at different points in time. These digital medical images may be stored into a digital storage device (e.g., hard drive, network drive, cloud storage, RAM, etc.). The system may further receive as input any other metadata characterizing the patient's health at different points in time. The metadata may include information such as whether the inputted digital medical images were treated or untreated, and, for the treated slides, treatment information corresponding to the particular digital medical images. The information related to treatment for the individual may include information such as type of treatment, time that treatment occurred (e.g., dates used, times treatment occurred, date treatment began, and/or date treatment ended), and treatment dosage amount. Additionally the metadata may contain information about the individual such as age and sex. The metadata may also include patient synoptic reports that correspond to the individual's digital medical images. Last, the metadata may include information as to the data and time that a medical sample was created.

At step 554, the system may apply the machine learning system to the data received at step 552, outputting a representation for each time point given. The trained system from FIG. 5A, may be capable of replacing one or more missing modalities with a generic missing token as expected by the system.

At step 556, the system may then output one or more single vector embeddings per patient that may be used by a downstream module. For example, the treatment effectiveness module 135 and/or the treatment recommendation module 136 may be capable of receiving the vector embedding.

As described earlier (e.g., step 208), the system may be capable of analyzing the effectiveness of a prior treatment for one or more individuals. For example, this may be performed by the treatment effectiveness module 135. The treatment effectiveness module 135 may be capable of receiving as input digital medical images (e.g. WSI) from when one or more patients were previously treated. The treatment effectiveness module 135 may also be capable of receiving metadata indicating past treatments that correspond to the received digital medical images. This information may include, type of treatment, dates treatment began and ended, treatment time of day, and/or treatment dosage. Past treatments may include, but are not limited to, radiotherapy, chemotherapy, hormone therapy or other forms of therapy.

The treatment effectiveness module 135 may have the ability to assess the state of the tissue (as shown in the digital medical image) and to identify the treated regions of the image to be analyzed. This step may be performed manually using an annotation tool or automatically using AI (e.g., the salient region detection module 133). With respect to the inputted digital medical images, either the entire image or specific image regions may be considered treated. The treatment effectiveness module 135 may then run a severity system to determine the severity of the treated areas as discussed in more detail below. The treatment effectiveness module may predict a score related to diseased tissue being eliminated referred to as a disease elimination score. The treatment effectiveness module may further predict a score related to healthy tissues being damaged, referred to as a healthy tissue score. Finally, the system may determine an overall treatment effectiveness score that is based on the disease elimination score and healthy tissue score.

Two example approaches to using machine learning to create a treatment effectiveness region detector include: strongly supervised methods that identify precisely where the morphology of interest could be found and weakly supervised methods that do not provide a precise location as discussed in greater detail below.

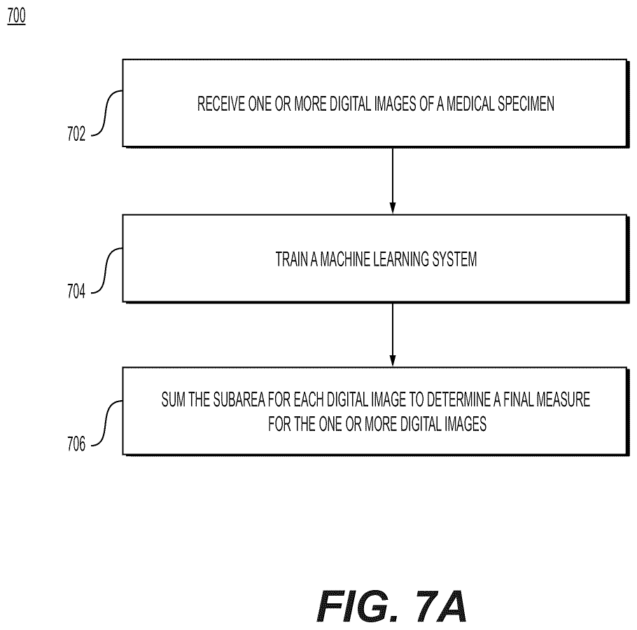

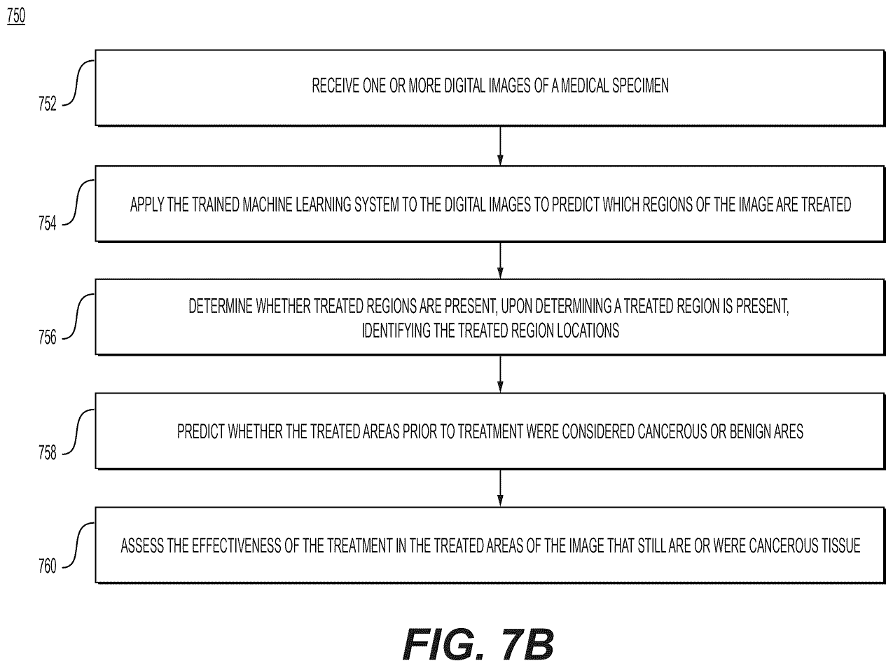

FIG. 7A is a flowchart illustrating an example method for training a system to determine a treatment effectiveness based on one or more a digital pathology image, such as, for example, a WSI, according to techniques presented herein. The processes and techniques described in FIG. 7A may be used to train a machine learning model to analyze one or more digital images to determine the effectiveness of one or more past treatments performed on a patient. The method 700 of FIG. 7A depicts steps that may be performed by, for example the treatment effectiveness module 135 of slide analysis tool 101 as described above in FIG. 1C. Alternatively, the method may be performed by an external system. Flowchart/method 700 depicts training steps to train a machine learning model as described in further detail in steps 702-706.

At step 702, the system may receive one or more digital medical images of a medical specimen (e.g., histology, CT, MRI, etc.) for one or more patients at various points of time into a digital storage device 109 (e.g., hard drive, network drive, cloud storage, RAM, etc.). For example, the system may receive a digital medical image for a specimen prior to treatment, at set time intervals during treatment, and after treatment. The system may further receive metadata that provides an indication of the presence or absence of the treated region (e.g., invasive cancer present, LVSI, in situ cancer, etc.) within the image. This may include information as to what type of disease may be present and/or the location of the disease.

Next, the system may identify regions/tiles of the received digital medical images that contain treatment effects. The system may perform this by splitting the digital medical image into smaller tiles. In another example, the system may perform this by using semantic segmentation of the digital medical images based on edge/contrast, segmentations via color differences, segmentations based on energy minimization, supervised determination by the machine learning model (e.g., the trained machine learning module in the salient region detection module 133 or the treatment effectiveness module 135), EdgeBoxes, etc. In another example, the metadata received at step 702 may include information regarding the regions/tiles of the digital medical image that contain treatment effect.

Next, the system may run a system to detect and quantify tumor infiltrating lymphocytes (TILs) within the digital medical images from step 702. The TILs may be white blood cells that may destroy tumor cells. The system may then assesses the viability of the TILs and creates a measure that is consumed by the system.

Next, the system may receive any other metadata characterizing the patient's health at each point in time (the point in time referring to the points at which the medical specimen may have been extracted for the digital medical images received at step 702). For example, measurements of a patient's overall health at each point in time may include a patient's blood pressure, temperature, level of paint/discomfort that a patient feels, etc.

At step 704, the system may train a machine learning system that is capable of identifying the treatment effectiveness of an individual based on digital medical images. The trained system may receive as input the digital medical images from step 702 and any additional other corresponding metadata (e.g., whether the slides are treated). This data may be received for multiple points of time for medical digital slides corresponding to a particular individual. The trained system may then be able to predict whether the treated region is present or not. The system may be trained utilizing either weak supervision or strong supervision in order to identify regions with morphology of interest. The system may be trained to output a total score defining the effectiveness of the past treatment (e.g., the overall treatment effectiveness score). The score may be based on the damage of the treatment to healthy tissue and the elimination of the diseased tissue.

For example, the system may be trained using weak supervision, where a machine learning model (e.g., multi-layer perceptron (MLP), convolutional neural network (CNN), Transformers, graph neural network, support vector machine (SVM), random forest, etc.) may utilize multiple instance learning (MIL) using weak labeling of the digital image or a collection of images. The labels of the training data may correspond to the presence or absence of a treated region. The trained ML system may be capable of receiving the embedding outputted from the embedding representation module 134 described above. The ML system may also be capable of receiving the treatment regimen and predicting/outputting the treatment effectiveness at each time step. The trained model may then be capable of predicting the treatment effectiveness at a future time step for an arbitrary treatment regimen.

For weakly supervised training, the system may receive the image or images (e.g., from step 702) and the presence/absence of the treated regions, but the exact location of the treated areas may not need to be specified. An input into training the system, in addition to whether the area was treated may also include whether the underlying areas were benign or cancerous. Next, the system may then predict whether the treated areas prior to treatment were considered cancerous areas or benign areas. The system may then be trained to output a score for the previously benign (e.g., the healthy tissue score) and for the previously cancerous regions (e.g., the disease elimination score) of the one or more digital medical images. First, the system may assess the effectiveness of the treatment in the treated areas of the image that still are or were cancerous tissue. The system may quantify the effectiveness of a treatment on a previously cancerous region by determining a disease elimination score. The disease elimination score may for example measure the decrease in a cancerous region in a quantifiable score. For example, a score of 0 may indicate a past treatment was not effective (e.g., cancer still present and/or cancer has spread) and a score of 10 may mean the treatment was very effective (e.g., all cancer eliminated), with varying gradations in between. In another example, the assigned score that the trained system outputted may be another type of metric such as binary, ordinal, continuous etc. The score may be assigned for particular subareas of the regions that previously included cancerous tissue. Next, the system may sum the cancerous region score for every subarea to determine a final disease elimination score for the entire digital medical image (e.g., a WSI). For example, this may be done by either taking a total overall score for the slide or determining an average score for the entire slide by averaging the score of each subarea. The outputted score may assesses/describe the severity of the treatment in the treated areas of the image that still are or were cancerous tissue.

Next, the system may quantify the effectiveness of a treatment by analyzing the previously benign regions of a digital medical image to determine a healthy tissue score. This score may analyze the effects of treatment on previously healthy tissues. The system may determine a benign region healthy tissue score for each digital medical image. The total score may define the severity of the disease within the tissue of the digital medical images. The healthy tissues score may be an averaged score defining the averaged healthy tissue grade for each tile/subsection of an image's healthy tissue. The healthy tissue may have been previously identified prior to the machine learning system outputting a healthy tissue score. For the healthy tissue score, a score of zero 0 may indicate the disease is not severe (benign tissue still present), and a score of 10 may indicate the disease is very severe (all benign tissue damaged). Alternatively, other types of metric such as binary, ordinal, continuous etc. may indicate the severity of whether benign tissues were damaged. This measure for every subarea is then summed to give a final measure for the entire digital medical image.

Finally, the system may determine a treatment effectiveness score that averages the healthy tissue score and the disease elimination score. This score may be used to rate a treatment effectiveness.

In another example, the machine learning model may be trained using strongly supervised training. For strongly supervised training, the image and the location of the treated regions may be received as input(s). Furthermore, information about whether the treated regions were malignant (e.g. cancerous) or benign may also be received. For 2D images, e.g., whole slide images (WSI) in pathology, these locations may be specified with pixel-level labeling, bounding box-based labeling or polygon-based labeling. For 3D images, e.g., CT and MRI scans, the locations may be specified with voxel-level labeling, using a cuboid, etc. or use a parameterized representation allowing for subvoxel-level labeling, such as parameterized curves or surfaces, or deformed template.

In one example, the machine learning model may be trained using bounding box or polygon-based supervision. This may include training a machine learning model (e.g., R-CNN, Faster R-CNN, Selective Search, etc.) using bounding boxes or polygons that specify the sub-regions of the digital image that are salient for the detection of the presence or absence of the treated areas.

In another example, the machine learning model may be trained utilizing pixel-level or voxel-level labeling (e.g., a semantic or instance segmentation). This may include training a machine learning model (e.g., Mask R-CNN, U-Net, Fully Convolutional Neural Network, Transformers, etc.) where individual pixels/voxels are identified as being salient for the detection of the continuous score(s) of interests. Labels could include in situ tumor, invasive tumor, tumor stroma, fat, etc. Pixel-level/voxel-level labeling can be from a human annotator or from registered images that indicate saliency.

During training, the machine learning system may be given input labels or segmentation masks describing cancerous or benign regions. These input labels and segmentation mask may be for digital medical images prior to and after treatment. The system may then be trained to predict whether the treated areas prior to treatment were considered cancerous areas or benign areas. Additionally, if the machine learning model received the cancerous and benign treated areas during training, the trained system also takes receive as input the disease elimination score, the healthy tissue score, and the overall treatment effectiveness score. For example the disease elimination score may be a value from 0 to 10 where a measure of 0 is not effective (cancer still present), 10 is very effective (all cancer eliminated). The disease elimination effectiveness score another type of metric such as binary, ordinal, percentage, or continuous etc. This disease elimination effectiveness score may be for each subarea of a particular inputted slide. Additionally, the system may receive the overall score (e.g., the combined score of the subareas discussed below).

Last, at step 706, the system may be trained to determine a final measure (e.g., an effectiveness score) for each of the inputted digital medical images. This may be done by averaging or compiling the scores of each subarea within the inputted particular slides. The determined overall treatment effectiveness score may then be compared to the overall treatment effectiveness score provided with the training slides to help further train the system. The trained system may then be saved to one or more storage devices such as storage devices 109.

FIG. 7B is a flowchart illustrating an example method for using a system to determine a treatment effectiveness based on one or more a digital pathology image, according to techniques presented herein. The exemplary method 750 (e.g., steps 752-760) of FIG. 7B depicts steps that may be performed by, for example, the treatment effectiveness module 135. These steps may be performed automatically or in response to a request from a user (e.g., a pathologist, a department or laboratory manager, an administrator, etc.). Alternatively, the method 750 may be performed by any computer process system capable of receiving image inputs such as device 1200 and capable of storing and/or executing the trained system described in FIG. 7A.

At step 752, the trained system may receive one or more digital medical images into a digital storage device (e.g., hard drive, network drive, cloud storage, RAM, etc.). The trained system may break each digital image into sub-regions using any of the techniques discussed within this application.

At step 754, the trained system may be applied to the tiles of the inputted digital medical images from step 752.

At step 756, the trained system may first predict which regions of the image have previously been treated. If the trained system determines that no treated regions are present, the system may output notification that no treatment was performed and that no treatment effectiveness score is available. If the trained system determines a region of the image has been previously treated, the trained system may continue to analyze the inputted digital medical slides. In one example, the system may receive metadata corresponding the inputted slides from step 752 noting whether each slide had been previously treated.

At step 758, if treated regions are present, the system may identify the location of treatment and flag them. Flagging them may including determining the pixel location of the region or of the boundary of the region. The trained system may identify the treated region variety of methods, including but not restricted to: running the machine learning model on image sub-regions to generate the prediction for each sub-region or using machine learning visualization tools to create a detailed heatmap, etc., and then extracting the relevant regions.

A step 758, the trained system may then predicts whether the treated areas were considered cancerous areas or benign areas prior to treatment.

Last, at step 760, the trained system may assess the effectiveness of the treatment in the treated areas of the image. This may include determining a treatment effectiveness sub-score for each tile and an overall treatment effectiveness score for each of the inputted images from step 750 that had a treatment applied. The system assesses the effectiveness (e.g., the disease elimination score) of the treatment in the treated areas of the image that still are or were cancerous tissue. The system quantifies the effectiveness into a measure e.g. 0 not effective (cancer still present), 10 very effective (all cancer eliminated), or another type of metric (binary, ordinal, continuous etc.). This measure for every subarea is then summed to give a final disease elimination score for the entire image/WSI. The system assesses the severity of the treatment in the treated areas of the image that still are or were benign tissue. The system quantifies (e.g., the healthy tissue score) the severity into a measure e.g. 0 not severe (benign tissue still present), 10 very severe (all benign tissue damaged), or another type of metric (binary, ordinal, continuous etc.). This measure for every subarea is then summed to give a final healthy tissue score for the entire image/WSI. Finally, the system may determine an overall treatment effectiveness core that is a combination of the disease elimination score and the healthy tissue score for each of the inputted images. This score may indicate the overall effectiveness of a previous treatment. In some examples, when the score passes a threshold value, the past treatment may be referred to as effective and a new treatment may not be recommended. If the score is below the threshold value, the system may then determine that the treatment recommendation module 136 should be utilized to determine a new treatment for the one or more patients.

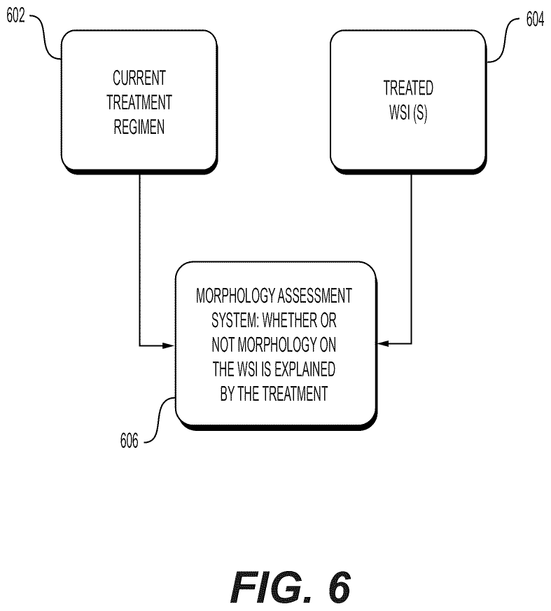

FIG. 6 displays an example use case of the of the treatment effectiveness module 135. The treatment effectiveness module 135 may receive as input treated digital medical images (e.g., treated WSI(s) 604) and a current treatment regimen 602. The current treatment regimen 602 may include metadata describing information related to past treatment such as treatment type, treatment dosage, and dates and times that treatment was applied. The treatment effectiveness module 135 may then apply the trained system to analyze whether or not the usage of multiple drugs together has created a unique morphology in which individual drugs alone may have created, at the morphology assessment system 606.

As described earlier (e.g., step 210), the system may be capable of analyzing digital medical images and providing a treatment recommendation based on the digital medical images for one or more individuals. This may be performed by the treatment recommendation module 136. The treatment recommendation module 136 may receive as input a digital medical image with a known tissue type and metadata noting whether or not the slide was treated. The treatment recommendation module 136 may assess the state of the tissue and recommend a treatment regimen. For example, the assessment may include frequency and amounts/dosages of a single or potential combination of drugs/treatments (e.g., from a known set of drugs/treatments used to treat the particular tissue type being analyzed) for the patient from which the slide was obtained. The module may also incorporate spatial characteristics of the salient tissue into the prediction. Two exemplary ways that the treatment recommendation module 136 may be trained using spatial characteristics include end-to-end and a two-stage prediction system. An end-to-end system may be trained directly from the input image whereas the two-stage system may first extract features from the image and then uses machine learning methods that can incorporate the spatial organization of the features.

The treatment recommendation module 136 may receive one or more digital medical images as input. In one example, the inputted digital medical images may include sets of images, wherein each set is for an individual patient. The sets may include digital medical images of the same one or more medical specimen at various times. The system may further receive metadata associated with the inputted digital medical image. In one example, the metadata may include time stamps and metadata to distinguish which medical digital images belong to the same individual and what time/location the sampling of the medical image took place. Further, the metadata may include the tissue type for the inputted images. In another example, the metadata may include list of past treatments and dosages of treatment for each medical slide over time. The metadata may further include clinical data about the patient over time, such as past treatment dosage levels (if any), time between past treatments, and genomic information for the one or more individuals associated with the digital medical images inputted. The treatment recommendation module 136 may receive the inputs discussed as embeddings from the embedding representation module 134 as discussed earlier. The treatment recommendation module 136 may output recommended dosage level for a current treatment each individual associated with the inputted digital medical images. Alternatively, the treatment recommendation module 136 may recommend that any alternative treatments should be used because the patient is not responding. In this case, the system may optionally provide the alternative treatment to be used. The outputs from the treatment recommendation module may be outputted through the output interface 137. Further, the treatment recommendation module 136 may output the received digital medical images with visualize regions of tissue on the digital images that are most effected/changed by the treatment, with quantifications of how effective the treatment was on these regions (i.e., “displays light effects of treatment” or “displays heavy effects of treatment”). This output may provide further information related to the effect of the treatment.

The slide analysis tool 101 may include or perform tasks such as, at each time point, construct a “universal embedding” that takes digital images from different modalities and can handle missing data along with the past treatment at that stage as input. This may be performed by the embedding representation module 134 as discussed above. If a particular modality of data is missing at a given time point, the system may interpolate the missing modality of data and construct the “universal embedding” with that interpolate data. The embeddings may then be inputted into the treatment recommendation module 136 at which point a trained system (e.g., a transformer or RNN) may aggregate these embeddings to determine the new treatment dosage. The treatment recommendation module 136 may utilize a generative method that estimates, given a treatment level, what is predicted to happen to the tissue, and then these outputs may be used to determine how much damage there was to the disease versus healthy tissue, in an iterative manner. This information may then be utilized to determine whether to recommend a new treatment for one or more individual.

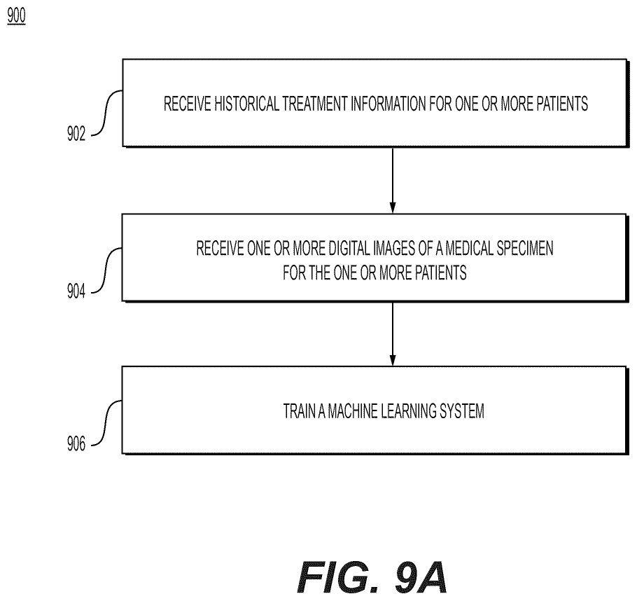

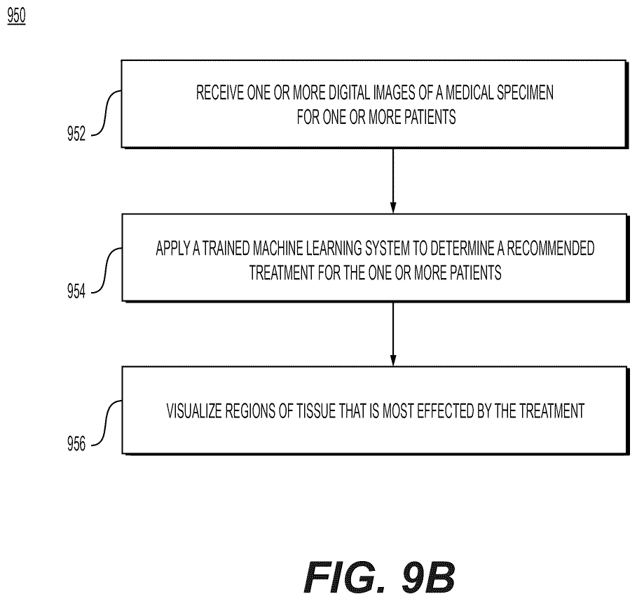

FIG. 9A is a flowchart illustrating an example method for training a system to determine a treatment recommendation based on one or more a digital pathology image, according to techniques presented herein. The processes and techniques described in FIG. 9A may be used to train a machine learning model to analyze one or more digital images to determine a treatment recommendation for one or more patients. The method 900 of FIG. 9A depicts steps that may be performed by, for example the treatment recommendation module 136 of slide analysis tool 101 as described above in FIG. 1C. Alternatively, the method may be performed by an external system. Flowchart/method 900 depicts training steps to train a machine learning model as described in further detail in steps 902-806.

At step 902, the system may first receive historical treatment metadata. This date may be received as either an electronically documented text paragraph, structured data or numbers stored into a digital storage device 109 (e.g., hard drive, network drive, cloud storage, RAM, etc.) and accessed via the network 120 including the hospital servers 122, research lab server 124, laboratory information system 125, clinical trial servers 123, physician servers 121 or other digital system. In one embodiment, the system may receive the historical treatment metadata in the form of an embedding imported from the embedding representation module 134.

At step 904, the system may receive one or more digital medical images for one or more patients into a digital storage device 109 (e.g., hard drive, network drive, cloud storage, RAM, etc.). The digital medical images may each correspond to information from the gross description provided at step 902. Further, the medical images may include metadata that contains the date and/or time that the medical specimen were sampled and converted to digital medical images. The system may receive auxiliary non-image input variables such as body temperature or external environmental temperature. Each image may be paired with relevant output information from the treatment regimen, e.g. the drugs used for treatment as well as respective amounts and frequencies corresponding to the digital medical image.

As will be described in greater detail below, the system may be trained using traditional regression (when the treatment regimen for a particular drug involves continuous numbers, e.g. 40, 50, 70 gray) or ordinal regression (when the treatment regimen for a particular drug involves whole numbers, e.g. 1×, 2×, 3× tablets). Multiple treatments with varying dosages/amounts may be provided as a treatment recommendation.

Additionally, the system may also receive patient information such as age, ethnicity, ancillary test results, etc. to stratify and split the system for machine learning. Additional information may also be ingested such as gross information and the watchful waiting time frame. Biomarkers such as genomic/epigenomic/transcriptomic/proteomic/microbiome information may also be ingested, such as point mutations, fusion events, copy number variations, microsatellite instabilities (MSI), or tumor mutation burden (TMB).

The salient region detection module may be used to identify the saliency of each region within the image and exclude non-salient image regions from subsequent processing. This may be performed on the inputted digital medical slides from step 904 by the salient region detection module 133. The treatment effectiveness module 136 may be utilized to quantify the extent of treatment effects on the regions identified by the salient region detection module 133.

At step 906, the treatment recommendation module 136 may train a machine learning system to predict one or more treatment regimen for one or more patients. To incorporate the spatial information, the coordinates of each pixel/voxel can optionally be concatenated to each pixel/voxel. Alternatively, the coordinates can optionally be appended throughout processing (e.g., by a CoordConv algorithm). Alternatively, the machine learning algorithm could take spatial information into consideration passively by self-selecting regions in the input to process. If patient information (e.g., age) and or genomic/epigenomic/transcriptomic/proteomic/microbiome is also used as an input in addition to medical image data, then that can be fed into the machine learning system as an additional input feature. If any treatment effects are quantified by the treatment effectiveness module, then that can be fed into the machine learning system as an additional input feature. Machine learning systems that could be trained include but are not limited to a CNN/CoordConv/Capsule network/Random Forest/Support Vector Machine/Transformer trained directly with the appropriate loss function.

FIG. 9B is a flowchart illustrating an example method for using a system to determine a treatment recommendation based on one or more a digital pathology image, according to techniques presented herein. The exemplary method 950 (e.g., steps 952-756) of FIG. 7B depicts steps that may be performed by, for example, the treatment recommendation module 136. These steps may be performed automatically or in response to a request from a user (e.g., a pathologist, a department or laboratory manager, an administrator, etc.). Alternatively, the method 950 may be performed by any computer process system capable of receiving image inputs such as device 1200 and capable of storing and/or executing the trained system described in FIG. 9A.

At step 952, the system may first receive one or more digital medical images of pathology specimens from an untreated or treated patient (e.g., histology, cytology, etc.) into a digital storage device 109 (e.g., hard drive, network drive, cloud storage, RAM, etc.). In one example, the received digital medical images may be first input into the embedding representation module 134 and the information may be received by the treatment recommendation module 136 as an embedding.

At step 954, the trained system (e.g. from step 906) may be applied to the received digital medical images. The trained system may predict a score that is an ordinal values, integers or real numbers. The system may further display these predictions in a viewing platform (e.g., by output interface 137), at step 956, or store them digitally (e.g., in storage devices 109). In addition, trained AI system may predict on to a digital medical image where the identified conditions are located. This may be displayed as by a heatmap, bounding box, pixel outline, or other representation. A user may have the option to not display the identified location, but instead to have the information displayed as a written description either near the image or as a separate output.

The trained system may also attribute the explanation for why a drug is given with a specific dosage and frequency. For example, if multiple digital medical images are input for a given deceased individual, it may rank them for each output in terms of providing the supporting evidence for that cause (e.g., by analyzing the level of positive output activity of a neural network when processing that image) and then indicate on the image the location of that evidence (e.g., using class activation maps, GradCAM, etc.).

The system may display the information (e.g., to a pathologist through output interface 137) and/or save the information to one or more electronic storage devices 109 such as a digital evidence and forensics system.

In another example, if the system detects a cause of death that is of potential legal concern, the system may alert/notify alert law enforcement or another personnel.

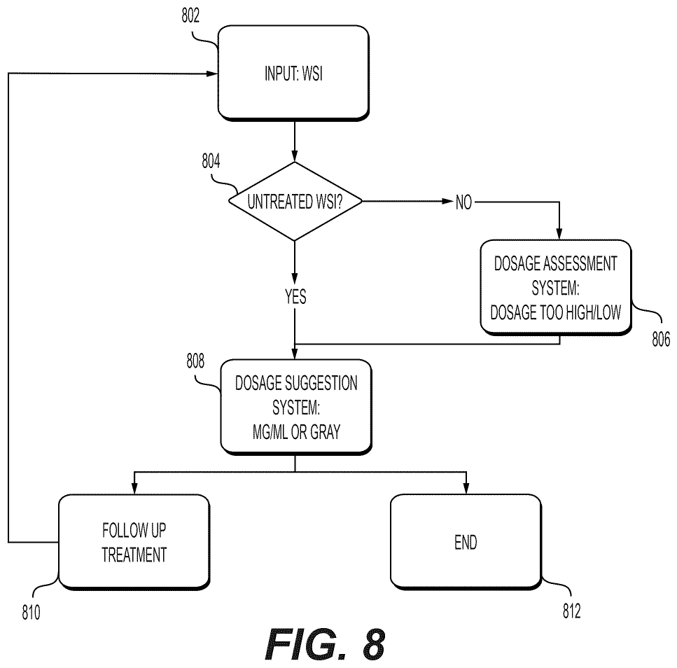

FIG. 8 is a flowchart illustrating an exemplary process for using a trained system (e.g., the treatment recommendation module 136) to determine a treatment recommendation based on one or more digital pathology images, according to techniques presented herein.

At step 802, the trained system may receive one or more inputted digit medical images (e.g., WSIs). At step 804, the system may determine whether the inputted digital medical images have previously been treated. This may be determined by additional input metadata containing information as to whether a slide has been treated or may be determined by the trained system.

At step 806, if the trained system has determined a slide was previously treated, the system may perform analysis to determine whether the previous treatment is effective. At step 806 the system may, determine whether the previous treatment was ineffective (e.g., had a low effectiveness score). If the system determines that the dosage has been effective (e.g., an appropriate effectiveness score), the digital medical image may output that no treatment recommendation is suggested.

At step 808, the untreated slides and slides that have been determine to have an inadequate dosage may be analyzed by the trained system of the treatment recommendation module 136. The system may output a dosage suggestion such as an amount and frequency of a particular dosage for the patient to utilize as future treatment.

In one example, the system may perform follow up treatment 810, where the digital medical images are re-inputted into the system at a later time and reanalyzed to determine whether the treatment is effective and/or whether an additional treatment recommendation update is suggested.

In another example, the system may end at step 812 by outputting a dosage type and suggestion to the one or more users. This may be outputted by output interface 137.

Examining the treatment recommendation module 136, the trained system may be capable of determining a dosage assessment and outputting an updated suggested dosage. This analysis may be performed after receiving as input either a treated, untreated slide, or a collection of treated or untreated slides. The system may output an amount/dosage and frequency for a single or potential combination of drugs. These suggestions may be given in gray or mg/mL, for example.

Additionally, for a treated slide or a collection of treated slides, an associated system may assess the effectiveness of a given treatment (e.g., amount/dosage and frequency for a single or potential combination of drugs) and provide an updated suggestion for amount/dosage.

Based on the morphology of the tissue and the drug treatment, a regression or ordinal classification system may be chosen. For certain drugs the system may use regression and recommend values such as 40, 50, or 70 gray. For fixed dosage formats such as tables, the system may use ordinal classification and suggest, 1×, 2×, 3×, 4× of tablets (or a multiple of the milligrams).

The system may help influence drug administration. Whether treatment is given 4 or 5 times a week and whether it is 1.5 or 2 gray per treatment.

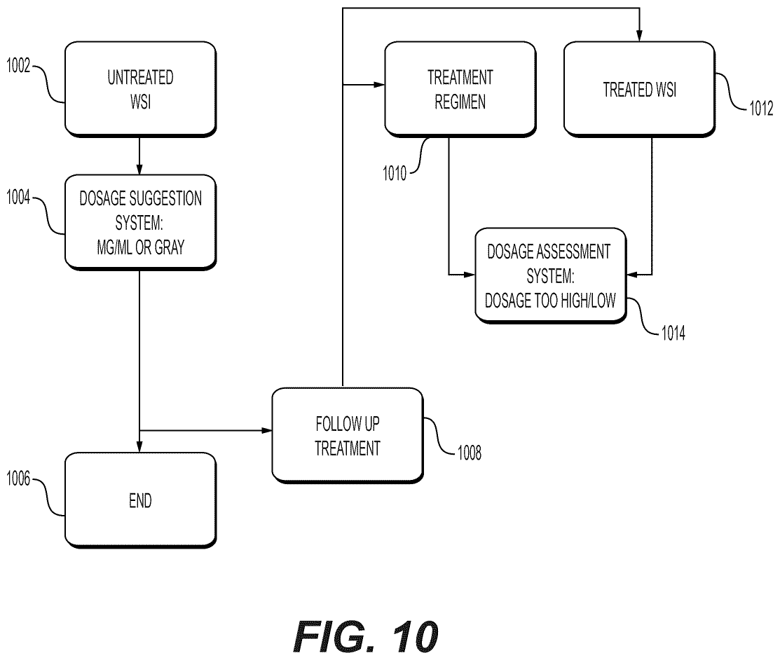

FIG. 10 is a flowchart illustrating an exemplary process for using a trained system to determine a treatment recommendation based on one or more digital pathology images, according to techniques presented herein. This may display an exemplary embodiment of the system that outputs a treatment dosage.

In FIG. 10's exemplary flowchart, the system (e.g., the treatment recommendation module 136) may first receive previously untreated digital medical images at step 1002. The trained system may then output a suggested dosage at step 1004. In one example, the system may end at step 1006. In another example, a follow up treatment may be performed at step 1008. The follow up treatment at step 1008 may include providing the suggested dosage to the patient. Next, at step 1010 and 1012, digital medical images of the same individual may be provided to the trained system in addition to metadata corresponding to the treatment provided to the patient. Last, at step 1014, the system (e.g., the treatment effectiveness module 135) may be utilized to determine whether the suggested treatment is effective.

The system described herein may include multiple use cases.

In one example, the trained system (e.g., slide analysis tool 101) may attribute morphological changes to one or more treatment regimens. In this example of the system, the system may receive a treated slide or collection of treated slides. Additionally the system may receive as input the current treatment regimen used for the sampled patient. Next, the system may assess whether the morphology identified on the slide(s) can be attributed to the treatment regimen. The assessment may be in the form of a binary classification (e.g., yes if the morphology can be attributed to the treatment, or no if it cannot). Additionally, a heat map may be shown to explain why the classification was made, highlighting which particular regions of the slide(s) indicated that the treatment explained the morphology sufficiently.

The system could be extended to quantify the treatment effects on the areas highlighted by the heat map. If a CNN is being used to jointly consider a tile from the treated WSI along with the treatment regimen, a heat map could be produced and given to a downstream model that processes the heat map for that tile and quantify the treatment, e.g., into bins of severity of the treatment. For example, the downstream model may output that the tile exhibits no treatment effects, light treatment effects, or heavy treatment effects. It may also output that there are/are not signs of treatment effects. These heat maps may then be displayed on the whole WSI and aggregated to give an assessment of how well the treatment worked. FIG. 6 discussed above depicts example flow diagram for a morphology assessment system.

In another use, the system (e.g., slide analysis tool 101) may be utilized to analyze whether tissue removal should be a suggested treatment. There may not be a traditional way to determine when tissue should be remove. Techniques disclosed herein may be used to assess and recommend future biopsies and resections. Input into the system may be a WSI from a biopsy or resection, gross information, duration of watchful waiting period other treatment and patient information. If the input is a WSI and a watchful waiting time frame, the system may suggest shorter or longer time frames. Given information from the gross report (such as the weight of the tissue in grams, margin information) and a WSI, the system may also suggest removing more or less tissue in a resection procedure. If the input is a WSI, the system may recommend a time frame when a follow up resection should be done and how much tissue should be removed.

In another use, the system (e.g., slide analysis tool 101) may be utilize to determine chemotherapy treatment for one or more individuals. This may include determining a type of chemotherapy therapy, a preferred dosage, and a schedule for when to administer the dosages. Given an untreated digital medical images or a collection of untreated digital medical images, the system may suggest an amount/dosage and frequency for a single or potential combination of chemotherapy drugs. These dosage suggestions may be output as gray or mg/mL, for example.

Additionally, for a treated digital medical images or a collection of treated digital medical images, an associated system (e.g., the treatment effectiveness module 135) could assess the effectiveness of a given treatment (i.e., amount/dosage and frequency for a single or potential combination of drugs) and provide an updated suggestion for amount/dosage.

Based on the morphology of the tissue and the drug treatment, a regression or ordinal classification system will be chosen. For fixed dosage formats such as tablets, the system will use ordinal classification and suggest, 1×, 2×, 3×, 4× of tablets (or a multiple of the milligrams).

Additionally, the system may output a drug category. This may be in addition to or in replacement of outputting an individual drug or set of drugs. For example, the system may output drug categories such as Alkylating agents, Antimetabolites, Plant alkaloids, Antitumor antibiotic, etc.

Additionally, the system may also output recommendations on how the recommended drug should be administered. For example, the system may suggest the individual intake the drug by oral, intravenous, or other methods.

In another use, the system (e.g., slide analysis tool 101) may be utilized to determine one or more radiation therapy treatments. This may include determining a type of radiation therapy, a preferred dosage, and a schedule for when to administer the dosages. Given an untreated digital medical image or a collection of untreated digital medical images, the system (e.g., the treatment recommendation module 136) may suggest an amount/dosage and frequency for a single or potential combination of radiation treatments. The recommendations may be output as units of gray, for example.

Additionally, for a treated digital medical images or a collection of treated digital medical images, an associated system (e.g., the treatment effectiveness module 135) may assess the effectiveness of a given treatment (e.g., amount/dosage and frequency for a single or potential combination of treatments) and provide/output an updated suggestion for amount/dosage. The system may for example output the amount/dosage through the output interface 137.

Based on the morphology of the tissue and the drug treatment, a regression or ordinal classification system may be chosen. For certain treatments the system may use regression and recommend values such as 40, 50, 70 gray.

In one example, the system (e.g., the output interface 137) may output information related to drug administration. The system may output how many and what days of the week to for an individual to intake a drug. The system may further output the time of day and particular dosage. For example, an output may be whether treatment is given four or five times a week and whether it is 1.5 or 2 gray per treatment. The system may also suggest optimizing the intervals between treatments. This may include suggesting a preferred amount of time between dosage intake such as suggesting the dosage be administered exactly twenty four hours apart.

The system may also use a temporal suggestion system. This may include the system receiving digital medical images of the same medical specimen for the same individual at different time intervals. Given a digital medical image treated at time point t_0 and a slide from the removed tissue at a later time point t_1, the system assesses whether the tissue should have been removed earlier.

In another use, the system (e.g., slide analysis tool 101) may be utilized to determine one or more hormone therapy treatments. This may include determining a type of hormone therapy, a preferred dosage, and a schedule for when to administer the dosages. In some cases, the growth of cancer may be attributed to hormones that attach to the cancer cells and allow for cancer to grow. Hormone therapy may work by slowing or stopping the growth of these types of cancers, such as prostate and breast cancer. Accordingly, it may be useful for the system to analyze digital medical images and to recommend/output hormone therapy regimens.

An input may be an untreated or treated digital medical images or a collection of digital medical images. If the patient was previously treated, an associated system (e.g., the treatment effectiveness module) may assess the effectiveness of a given treatment (i.e. amount/dosage and frequency for a single or potential combination of drugs) and provide an updated suggestion for amount/dosage. The system may suggest an amount/dosage and frequency for a single or potential combination of hormone therapy drugs and additional drugs. These suggestions may be given in mg/ml, for example. Based on the morphology of the tissue and the drug treatment, a regression or ordinal classification system may be chosen. For other fixed dosage formats such as tablets, the system may use ordinal classification techniques as discussed above and may output a tablet or milligram suggestion for each dosage (e.g., 1×, 2×, 3×, 4× of tablets).

For example, the system (e.g., the slide analysis tool 101) may receive digital medical images of breast tissue samples. The system may analyze the inputted digital medical images and output a treatment recommendation of drugs that block estrogen receptors, such as tamoxifen, toremifene, or fulvestrant, as well as drugs that lower estrogen levels. Given that a long-term therapy schedule may entail a drug that blocks estrogen receptors for 2-3 years, followed by a drug that lowers estrogen levels for 5-10 years, the system may recognize the morphology corresponding to tissue samples treated with e.g. tamoxifen, and recommend a follow-up regimen of a drug that lowers estrogen level, e.g. an aromatase inhibitor.

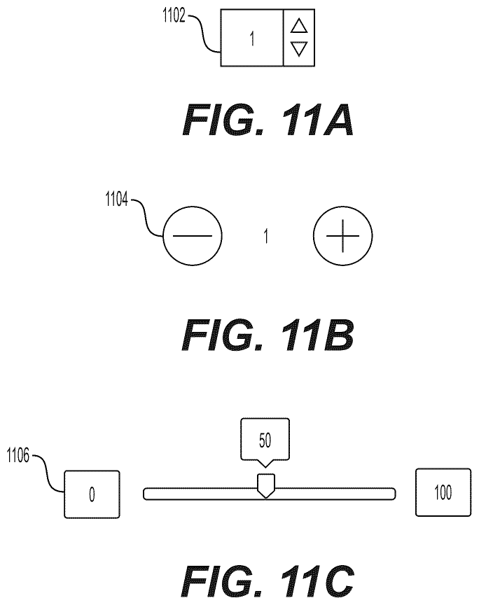

In another use, the system (e.g., the tissue viewing platform 100) may be capable of providing a visual output of the effect on tissue based on a varying potential treatment types and dosages. The system may include one or more user interfaces (e.g., a UI). The user interface may allow for a user (e.g., a pathologies) to enter hypothetical dosages and the system may be capable of outputting a visual extrapolation (e.g., markings) of how dosage could affect the tissue of one or more digital medical images. For example, if the system receives as input one or more untreated or treated digital medical images and a treatment regimen, a generative model (e.g. a GAN of treatment recommendation module 136) may extrapolate how the slide would look after the treatment has been applied. A user may access and utilize a user interface (e.g., a sliding device such as the user interface depicted in FIG. 11C) to select a period of time. The selected period of time may be the period of time that a treatment may be theoretically applied to one or more individuals. A user may further access and utilize a user interface to select a dosage for a potential treatment. An exemplary use of this may be that a user selects a time period of one month and a dosage 1 tablet a day for a particular treatment, and then the system may be capable of outputting a digital medical images that displays the predicted effects of utilizing one or more treatments recommendations for one month. In some examples, the treatment recommendation module 136 may output a recommended treatment. A user (e.g., a pathologist) may then be able to enter a period of time and dosage for the recommended treatment and the system may output a digital medical image that predicts the effects of the treatment of the digital medical images. This may allow for a pathologist to then alter a recommended treatment based on the predicted effects on the digital medical images. For example, a pathologist may see that a particular treatment may have an excessive effect and thus lower the recommended treatment dosage.

The UI may have a fixed number of known drugs specific to the particular tissue type in consideration, and may allow for a user to change the amount of each drug. FIG. 11A-11C display exemplary user interfaces that a user (e.g., a pathologist) may utilize to select a treatment amount. FIG. 11A shows a numeric stepper 1102 to set milligrams to be given to a patient. FIG. 11B shows an example input for receiving numbers 1104 (e.g., number of tablets). FIG. 11C shows a slider 1106 (e.g., to set a dosage).

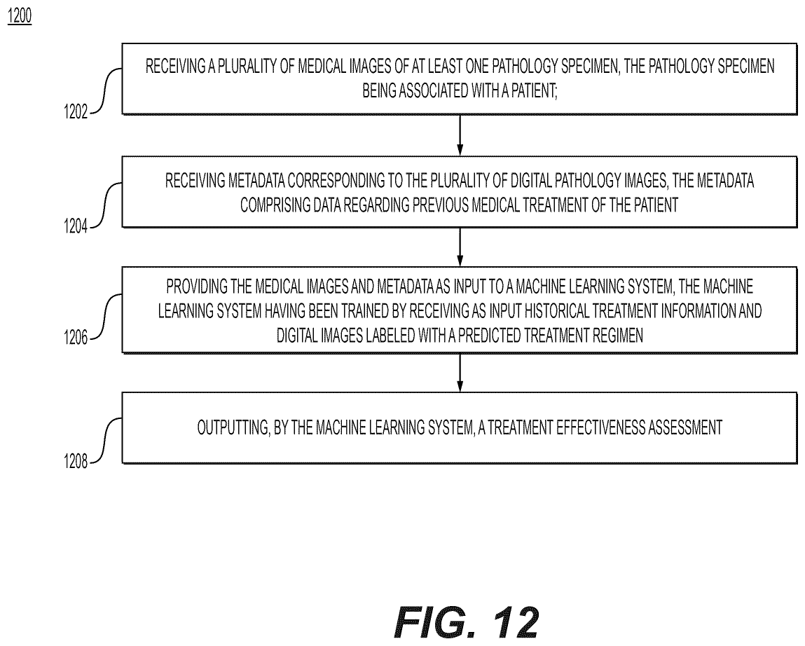

FIG. 12 is a flowchart illustrating an example method for determining a treatment recommendation for one or more users. At step 1202, a plurality of medical images of at least one pathology specimen, the pathology specimen being associated with a patient may be received.

At step 1204, the system may receive metadata corresponding to the plurality of digital pathology images, the metadata comprising data regarding previous medical treatment of the patient.

At step 1206, the system may provide the medical images and metadata as input to a machine learning system, the machine learning system having been trained by receiving as input historical treatment information and digital images labeled with a predicted treatment regimen.

At step 1208, the system may output by the machine learning system, a treatment effectiveness assessment.

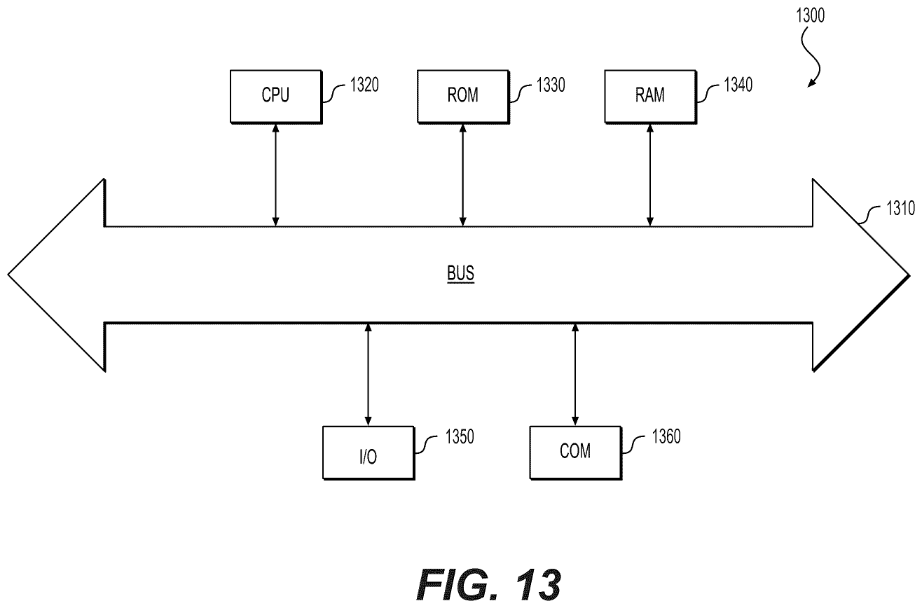

FIG. 13 depicts an example of a computing device that may execute techniques presented herein, according to one or more embodiments. As shown in FIG. 13, device 1300 may include a central processing unit (CPU) 1320. CPU 1320 may be any type of processor device including, for example, any type of special purpose or a general-purpose microprocessor device. As will be appreciated by persons skilled in the relevant art, CPU 1320 also may be a single processor in a multi-core/multiprocessor system, such system operating alone, or in a cluster of computing devices operating in a cluster or server farm. CPU 1320 may be connected to a data communication infrastructure 1310, for example a bus, message queue, network, or multi-core message-passing scheme.

Device 1300 may also include a main memory 1340, for example, random access memory (RAM), and also may include a secondary memory 1330. Secondary memory 1330, for example a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 1330 may include similar means for allowing computer programs or other instructions to be loaded into device 1300. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 1300.

Device 1300 also may include a communications interface (“COM”) 760. Communications interface 1360 allows software and data to be transferred between device 1300 and external devices. Communications interface 1360 may include a modem, a network interface (such as an Ethernet card), a communications port, a PCMCIA slot and card, or the like. Software and data transferred via communications interface 1360 may be in the form of signals, which may be electronic, electromagnetic, optical or other signals capable of being received by communications interface 1360. These signals may be provided to communications interface 1360 via a communications path of device 1300, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 1300 may also include input and output ports 1350 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

The foregoing general description is exemplary and explanatory only, and not restrictive of the disclosure. Other embodiments may be apparent to those skilled in the art from consideration of the specification and practice of the invention disclosed herein. It is intended that the specification and examples be considered as exemplary only.

1-20. (canceled)

21. A computer-implemented method for performing a clinical prediction, comprising:
i) retrieving input data via at least one communication interface of a processing device, wherein the input data comprises multiple different modalities of a patient;
ii) processing the input data by using the processing device, wherein the processing comprises generating embedding modality representations from the input data by using at least one trainable data embedder, wherein the processing comprises combining the embedding modality representations using at least one neural network thereby generating a clinical prediction; and
iii) generating an output of the clinical prediction by using the processing device.

22. The method according to claim 21, wherein the output of the clinical prediction comprises one or more of information about drugs for the patient, information about response to at least one specific treatment, information curating and/or completing patient data by predicting missing patient data points.

23. The method according to claim 21, wherein the method comprises at least one output step comprising providing the clinical prediction via at least one output interface, wherein an output of the trainable data embedder is a generic patient level embedding representation per modality or multiple instance embeddings for each modality.

24. The method according to claim 21, wherein the multiple different modalities of a patient comprise one or more of at least one histology tissue image, at least one whole side image of a biopsy, radiology images such as magnetic resonance imaging (MRI) and computed tomography (CT), genomic data, proteomics, patient clinical data.

25. The method according to claim 21, wherein the input data comprises at least one datapoint from each of the multiple different modalities, wherein the method comprises generating an embedding modality representation from each of the datapoints and generating from the embedding modality representations of the multiple different modalities the clinical prediction using the neural network.

26. The method according to claim 21, wherein the multiple different modalities are converted to embedding modality representations by the trainable data embedder and are then input into a second machine learning network that combines the embedding modality representations into a clinical prediction.

27. The method according to claim 21, wherein the multiple different modalities are converted to embedding modality representations by the trainable data embedder and then input into a transformer neural network that combines the embedding modality representations into a clinical prediction.

28. The method according to claim 21, wherein the trainable data embedder is a transformer neural network.

29. The method according to claim 21, wherein, depending on a respective modality of the input data, each modality is converted to the embedding modality representations by the trainable data embedder, wherein the embedding modality representations are input into a transformer neural network that combines the embedding modality representations into a clinical prediction.

30. A system for performing a clinical prediction, the system comprising:
at least one memory storing instructions; and
at least one processor configured to execute the instructions to perform operations comprising:
i) retrieving input data via at least one communication interface of a processing device, wherein the input data comprises multiple different modalities of a patient;
ii) processing the input data by using the processing device, wherein the processing comprises generating embedding modality representations from the input data by using at least one trainable data embedder, wherein the processing comprises combining the embedding modality representations using at least one neural network thereby generating a clinical prediction; and
iii) generating an output of the clinical prediction by using the processing device.

31. The system according to claim 30, wherein the output of the clinical prediction comprises one or more of information about drugs for the patient, information about response to at least one specific treatment, information curating and/or completing patient data by predicting missing patient data points.

32. The system according to claim 30, wherein the system comprises at least one output step comprising providing the clinical prediction via at least one output interface, wherein an output of the trainable data embedder is a generic patient level embedding representation per modality or multiple instance embeddings for each modality.

33. The system according to claim 30, wherein the multiple different modalities of a patient comprise one or more of at least one histology tissue image, at least one whole side image of a biopsy, radiology images such as magnetic resonance imaging (MRI) and computed tomography (CT), genomic data, proteomics, patient clinical data.

34. The system according to claim 30, wherein the input data comprises at least one datapoint from each of the multiple different modalities, wherein the system comprises generating an embedding modality representation from each of the datapoints and generating from the embedding modality representations of the multiple different modalities the clinical prediction using the neural network.

35. The system according to claim 30, wherein the multiple different modalities are converted to embedding modality representations by the trainable data embedder and are then input into a second machine learning network that combines the embedding modality representations into a clinical prediction.

36. The system according to claim 30, wherein the multiple different modalities are converted to embedding modality representations by the trainable data embedder and then input into a transformer neural network that combines the embedding modality representations into a clinical prediction.

37. The system according to claim 30, wherein the trainable data embedder is a transformer neural network.

38. The system according to claim 30, wherein, depending on a respective modality of the input data, each modality is converted to the embedding modality representations by the trainable data embedder, wherein the embedding modality representations are input into a transformer neural network that combines the embedding modality representations into a clinical prediction.

39. A non-transitory computer-readable medium storing instructions that, when executed by a processor, perform operations processing electronic medical images, the operations comprising:
i) retrieving input data via at least one communication interface of a processing device, wherein the input data comprises multiple different modalities of a patient;
ii) processing the input data by using the processing device, wherein the processing comprises generating embedding modality representations from the input data by using at least one trainable data embedder, wherein the processing comprises combining the embedding modality representations using at least one neural network thereby generating a clinical prediction; and
iii) generating an output of a clinical prediction by using the processing device.

40. The non-transitory computer-readable medium according to claim 39, wherein the output of the clinical prediction comprises one or more of information about drugs for the patient, information about response to at least one specific treatment, information curating and/or completing patient data by predicting missing patient data points.
